(19) United States
(12) Patent Application Publication
(10) Pub. No.: US 2019/0224048 A1
(43) Pub. Date: Jul. 25, 2019

(54) SYSTEMS AND METHODS FOR LOWERING INTRAOCULAR PRESSURE

(71) Applicant: THE TRUSTEES OF THE UNIVERSITY OF PENNSYLVANIA, Philadelphia, PA (US)

(72) Inventors: Richard Stone, Haverford, PA (US); Eydie Miller, Philadelphia, PA (US); Brandon W. Kao, Placentia, CA (US); Adarsh Battu, Fremont, CA (US); Rui Jing Jiang, Lexington, MA (US)

(73) Assignee: THE TRUSTEES OF THE UNIVERSITY OF PENNSYLVANIA, Philadelphia, PA (US)

(21) Appl. No.: 16/255,304

(22) Filed: Jan. 23, 2019

Related U.S. Application Data
(60) Provisional application No. 62/620,844, filed on Jan. 23, 2018.

(51) Int. Cl. A61F 9/007; A61L 31/02

(52) U.S. Cl. CPC A61F 9/00781; A61L 31/022

(57) ABSTRACT

Systems for lowering intraocular pressure of glaucoma patients are provided. An exemplary system of lowering intraocular pressure include an adjustable nanoscale plate structure to create an external reservoir. Methods of lowering intraocular pressure using such systems are also provided.

### PRIORITY CLAIM

This application claims priority to U.S. Provisional Application Ser. No. 62/620,844, filed Jan. 23, 2018, which is hereby incorporated by reference herein in their entireties.

### BACKGROUND

Glaucoma represents a group of eye diseases which can result in damage to optic nerves and vision loss. Glaucoma is the second leading cause of blindness in the US and world-wide. Risk factors for glaucoma can include increased pressure in the eye (i.e., intraocular pressure), increasing age, a family history of the condition, myopia, migraines, high blood pressure, and obesity. Despite multiple risk factors, one treatable parameter is to lower intraocular pressure. This can be achieved by eye drops, pills, or a combination of thereof. For patients with advanced disease, surgery to lower intraocular pressure can prevent blindness.

Certain drainage implant devices, placed into an eye, can lower intraocular pressure by increasing outflow of fluid from the eye. The success of such implants can relate to the surface area covered by the implant for drainage: The larger the implant surface, the greater the likelihood of successful outflow. However, an increased size of an implant can lead to a greater chance of complications. For example, certain drainage devices can require extensive tissue dissection and cause inflammatory immune responses after surgery, which commonly increase intraocular responses. Other complications can include corneal injury, which can result from mechanical contacts and micro-frictions between the implant and the tissues of the eye. Certain drainage devices have suboptimum success rates and/or complications.

Accordingly, there remains a need to develop improved drainage systems and techniques for lowering intraocular pressure to treat glaucoma.

### SUMMARY

The disclosed subject matter relates to techniques for the treatment of glaucoma by lowering intraocular pressure.

In some embodiments, the disclosed subject matter provides methods of lowering intraocular pressure by using a drainage device including a plate structure having a thickness between about 1 nm and about 1000 nm. An exemplary method can include modifying a geometry of the plate based on an eye condition of the subject, and securing a plate structure to the eye of the subject. For example, a shape of the plate structure can be modified based on an eye condition of a subject such as size and geometry of the eye. In certain embodiment, the securing can be performed by placing the plate structure between a sclera and a conjunctiva of the eye.

In some embodiments, the method can include inserting tubing into an anterior chamber of the eye to divert a portion of fluid from inside of the eye to an external reservoir created by the plate structure. The inserting can include inserting the tubing into an anterior chamber of the eye through a limbal region, a corneo-limbal region, or an anterior scleral region of the eye. The method can further include modifying a geometry of the tubing based on the eye condition of the subject. The geometry of the tubing can be modified to adjust fluid penetration and/or filtration.

In non-limiting embodiments, the method can include implanting the drainage device into the eye using an insertion device such as a forceps or a glide. For example, a nano plate structure can be held using a forceps and slide into a space between a sclera and a conjunctiva of an eye using a surgical glide. The surgical glide can keep the plate structure flat to avoid possibility of prolapse or trauma.

In certain embodiments, the disclosed subject matter provides a drainage device including a plate structure. The plate structure can have a thickness between about 1 nm and 1000 nm and create an external reservoir on a surface of an eye.

In certain embodiments, an exemplary plate structure can comprise at least one base plate in a first plane; and a plurality of out-of-plane rib plates forming at least one strengthening rib. The at least one strengthening rib can be formed such that there is no straight-line path extending through the height or width of the plate structure that does not intersect the at least one base plate and the at least one strengthening rib. In some embodiments, the plate structure can include a biocompatible material. For example, the biocompatible material can include aluminum oxide, hafnium oxide, silica, titanium nitride, titanium carbide, a derivative thereof, and a combination thereof.

In certain embodiments, the system can further include a tubing to divert a portion of fluid from inside of the eye to the external reservoir. An exemplary tubing can comprise a biocompatible polymer. In some embodiment, a shape of the tubing can be modified based on an eye condition of a subject such as size and geometry of the eye.

### BRIEF DESCRIPTION OF THE DRAWINGS

Further features and advantages of the present disclosure will become apparent from the following detailed description taken in conjunction with the accompanying figures showing illustrative embodiments of the present disclosure, in which:

FIG. 1 is a schematic illustration of an exemplary system implanted into an eye for lowering intraocular pressure.

FIG. 2 is a schematic illustration of an exemplary drainage device in accordance with the present disclosure.

FIG. 3 is a schematic illustration of an exemplary drainage device tailored to fit in subconjunctival space away from a scar tissue.

FIG. 4 is a schematic illustration of an exemplary drainage device geometry for broad filtration zone anterior to muscles,

FIG. 5 is a schematic illustration of an exemplary drainage device geometry for posterior placement.

FIG. 6 is a schematic illustration of an exemplary drainage device geometry for a posterior, broad filtration zone.

Throughout the figures, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments.

### DETAILED DESCRIPTION

The disclosed subject matter provides techniques for the treatment of glaucoma by lowering intraocular pressure. The subject disclosed subject matter relates to an implantable drainage device that is based on a plate structure to lower intraocular pressure in glaucoma patients.

The term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

A “subject” herein may be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

The term “biocompatibility” means as used herein refers to compatibility with living tissue or a living system by not being toxic, injurious, or physiologically reactive and not causing immunological rejection.

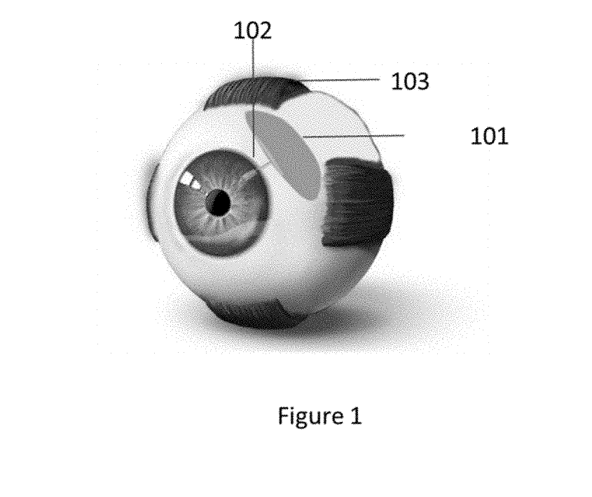

In non-limiting embodiments, the disclosed subject matter provides tubing. As shown in FIG. 1, the tubing 102 and a plate structure 101 can be inserted into an eye 103 to divert a portion of intraocular fluid to an external reservoir created by the plate structure. The distal end of the tubing 102 can be inserted into an anterior chamber of the eye through a limbal region, a corneo-limbal region, or an anterior scleral region of the eye. In certain embodiments, prior to the tubing insertion, the tubing can be sterilized to minimize postoperative complications and primed with balanced salt solution.

In some embodiments, the tubing can include a hollow cylindrical body with an inner diameter less than the outer diameter. The tubing can have a variety of lengths and diameters depending on source and intended use. For example, shape, length, inner diameter, and/or outer diameter of the tubing can be modified to allow fluid penetration and/or filtration for diverting a portion of fluid from an eye. A tubing can have an inner diameter up to 0.01 mm, 0.05 mm, 0.11 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1 mm, 2 mm, 3 mm, 4 mm, and 5 mm. In particular embodiments, the tubing 102 can have a diameter up to 0.5 mm. Further, a tubing 101 can have an outer diameter of at least 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1 mm, 2 mm, 3 mm, 4 mm, 5 mm, or longer.

In certain embodiments, the tubing can be a polymer tube having a lumen. As embodied herein, the polymer can be a biocompatible polymer. A biocompatible polymer does not produce toxin or harmful products and minimizes an immune response in biological systems. For example, and not limitation, suitable biocompatible polymers can include silicone rubber, polyethylene, polypropylene, poly(methyl methacrylate) (PMMA), poly(tetrafluoroethylene) (PTFE), polystyrene, polyethylcyanoacrylate, poly(vinyl chloride) (PVC), polyether ether ketone (PEEK), polyether sulfone (PES), polymer gels and combinations thereof. In certain embodiments, the biocompatible polymer can include a single type of polymer or a combination of different polymers, e.g., as a polymer blend and/or copolymer. In certain embodiments, the polymeric matrix can be one or more flexible polymers and/or one or more solid polymers. In non-limiting embodiments, the biocompatible polymer can include collagen type materials such as a Xen gel stent.

In accordance with the principles of the present invention, the tubing can be modified depending on intended use. The modification may take one of a number of forms, and comprise topographic features. For example, the tubing can include a filter within proximal and/or distal ends of the tubing to regulate the flow of intraocular fluid. The shape, length, inner diameter, and/or outer diameter of the tubing can be modified based on eye conditions of a subject. The tubing can be modified to adjust fluid penetration and/or filtration. In non-limiting embodiments, the tubing can include a cylindrical body with perforations.

In certain embodiments, the disclosed subject matter provides a plate structure 101. The plate structure can include base plates and rib plates forming at least one strengthening rib. The strengthening rib can be formed such that there is no straight-line path extending through the height or width of the plate structure that does not intersect the at least one base plate and the at least one strengthening rib. For example, a plate structure 101 can have a honeycomb structure. The term “plate structure,” as used herein, refers to a continuous structure. However, the term “plate structure” does not require that the structure be flat over its entirety.

In some embodiments, the plate structure 101 can include a plurality of base plates. The base plates can generally be characterized as residing in a single plane when the plate structure is disposed on a flat surface. However, plate structures in accordance with the disclosed subject matter can be flexible and may be disposed on curved surfaces or the like. Therefore, the base plates can be considered to reside in the same plane if they are in a single plane when the plate structure is disposed on a flat surface.

In certain embodiments, the plate structure can further include one or more strengthening ribs. In the plate structure, the strengthening ribs form a honeycomb pattern. However, those skilled in the art will understand that the disclosed subject matter is not limited to the honeycomb structure. For example, in accordance with other embodiments of the disclosed subject matter, the strengthening rib can form a basket-weave pattern. The strengthening ribs can prevent the plate structure from bending along a straight line. That is, the strengthening ribs can be formed such that there is no straight-line path across the plate structure that does not intersect at least one of the one or more base plates and at least one of the one or more strengthening ribs. In embodiments that do not avoid the existence of such straight lines, the bending stiffness of the plate can be reduced. The existence of such straight lines can cause the plate to fold easily along these straight lines.

In certain embodiments, the plate structure can be a nanostructure. For example, in accordance with embodiments of the disclosed subject matter, the plate structure can have a thickness of between about 1 nm and about 1000 nm, between about 1 nm and about 500 nm, between about 1 nm and about 250 nm, between about 1 nm and about 100 nm, between about 10 nm and about 100 nm, between about 20 nm and about 100 nm, between about 20 nm and about 75 nm, or between about 20 nm and about 50 nm. For example, the plate structure can have a thickness of about 5 nm, about 10 nm, about 15 nm, about 20 nm, about 25 nm, about 30 nm, about 35 nm, about 40 nm, about 45 nm, about 50 nm, about 75 nm, or about 100 nm.

In non-limiting embodiments, the plate structure in accordance with the disclosed subject matter can have a wide range of heights. The term “height,” as used herein, refers to the distance between the base plate and the top of the strengthening rib. In accordance with embodiments of the disclosed subject matter, the plate structure can have a height of between about 1 μm and about 100 μm, between about 1 μm and about 50 μm, between about 1 μm and about 30 μm, between about 5 μm and about 25 μm, and between about 5 μm and about 15 μm. For example, the plate structure can have a height of about 1 μm, about 5 μm, about 7 μm, about 9 μm, about 10 μm, about 11 μm, about 13 μm, about 15 μm, about 20 μm, about 25 μm, about 30 μm, about 50 μm, or about 100 μm.

In some embodiments, the plate structure in accordance with the disclosed subject matter can also have a wide range of lengths and/or widths. The term “lateral dimension,” as used herein, will refer to the length and width. In accordance with some embodiments, at least one lateral dimension of the plate structure can be between about 0.1 mm and about 15 mm, between about 1 mm and about 6 mm, between about 3 mm and about 6 mm, between about 4 mm and about 6 mm, or between about 4 mm and about 6 mm. For example, at least one lateral dimension can be about 5.9 mm. In accordance with other embodiments, at least one lateral dimension can be between about 1 mm and about 15 mm, between about 3 mm and about 15 mm, between about 5 mm and about 15 mm, or between about 10 mm and about 15 mm. For example, at least one lateral dimension can be at least about 9 mm. In some embodiments, the cellular structure of the plate structures can be invisible to the naked eye, and the cellular plate can instead be considered a metamaterial with effective properties determined not only by the properties of solid material that it is composed of, but its properties can be determined and/or affected also by the geometry of its cells. In some embodiments, plate mechanical metamaterials can exhibit novel properties that are not achievable with available solid and/or foam-like materials. For example, plate mechanical metamaterials can have the ability to sustain sharp bending deformations without sustaining damage. In some embodiments, plate mechanical metamaterials can have enhanced bending stiffness while exhibiting greatly reduced tensile stiffness, which cannot be achieved with planar structures regardless of thickness.

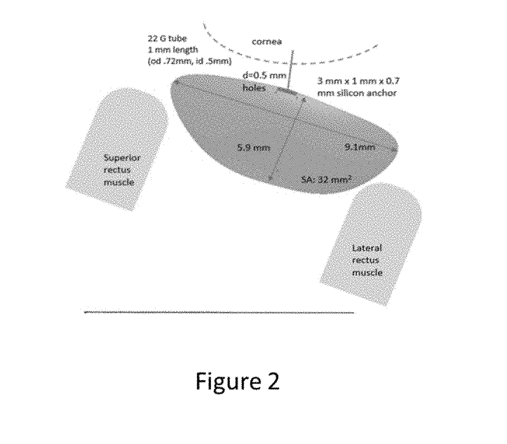

In an exemplary embodiment of the disclosed subject matter, as shown in FIG. 2, the plate structure can have a thickness between about 20 nm and about 75 nm and at least one lateral dimension between about 5 mm and about 15 mm.

While exemplary dimensions of plate structures are described, persons skilled in the art that these dimensions are provided for purposes of explanation and not limitation.

In accordance with embodiments of the disclosed subject matter, the plate structure can be composed of a plurality of cells. The cell includes a planar base portion and at least one out-of-plane portion. The term “planar,” as used herein, refers to the characteristic that the base portion resides in the same plane as the base portions of adjacent cells. The out-of-plane portion of cell includes vertical walls that are perpendicular to the base portion and horizontal walls that are parallel to the base portion but lie in a different plane. However, in accordance with embodiments of the disclosed subject matter, the out-of-plane portion need not be limited to vertical and horizontal walls. For example, the out-of-plane portion can include walls that extend from the base portion at a 45-degree angle.

In certain embodiments, the out-of-plane portion of cell can be connected to the out-of-plane portions of adjacent cells to form at least one strengthening rib. The strengthening rib can form a number of shapes, including a honeycomb shape. The cells of plate structure can have a common shape. In accordance with other embodiments of the disclosed subject matter, the shape of the cells need not be uniform. For example, the plate structure can include a first plurality of cells having a first shape and a second plurality of cells having a second shape. The first plurality of cells and the second plurality of cells can form an interconnecting pattern.

In some embodiments, the plate structure can be constructed from a variety of materials. For example, in accordance with an exemplary embodiment of the disclosed subject matter, the plate structure can be made of a biocompatible material such as silica (SiO₂), alumina (Al₂O₃), hafnium oxide (HfO₂), titanium nitride (TiNₓ), titanium carbide (TiC), derivative thereof, and a combination thereof. The plate structure material can also be, for example, a metal such as platinum or tungsten.

In certain embodiments, the plate structure in accordance with the disclosed subject matter can exhibit certain properties. For example, plate structures in accordance with the disclosed subject matter can have higher flexural stiffness as compared to planar films. For example, plate structures having the same thickness as planar structures have a much higher spring constant when used as cantilevers and/or doubly clamped beams. Similarly, plate structures having the same spring constant as planar structures can be significantly thinner. For example, plate structures in accordance with the disclosed subject matter can be at least about 20 times thinner, at least about 15 times thinner, at least about 10 times thinner, or at least about 5 times thinner than planar structures having the same flexural stiffness.

In some embodiments, plate structures in accordance with embodiments of the disclosed subject matter can be ultralight. For example, the plate structures can have a relative density on the order of about 10⁻⁴. The plate structures can also have an areal density on the order of 100 milligram per square meter. For example, the plate structures can have an areal density between about 100 mg/m² and about 1000 mg/m², or between about 10 mg/m² and about 100 mg/m².

In non-limiting embodiments, the plate structure in accordance with embodiments of the disclosed subject matter can also possess shape-recovering properties. In particular, the plate structures can be flexible. For example, plate structures in accordance with certain embodiments of the disclosed subject matter can be bent at a 90-degree angle without breaking and will return to their original shape. In accordance with certain embodiments of the disclosed subject matter, the plate structure can be bent at up to a 180-degree angle without breaking and will return to its original shape. The shape recovery property is attributed to the design of the plate structure.

In certain embodiments, two or more plate structures as disclosed herein can be stacked to form a composite material. For example, in accordance with one embodiment of the disclosed subject matter, two honeycomb structures can be fabricated as described herein above. After fabrication, the structures can be manually stacked to form a metamaterial. Alternatively, two or more structures can be fabricated in a pre-stacked arrangement. For example, the fabrication process can include additional deposition, masking, and etching steps so as to form two or more layers of atomic layer deposition (ALD), which when released can be in a desired stacked configuration.

In some embodiments, the structures can be stacked in a variety of configurations to achieve desired properties. For example, and not limitation, two structures can be stacked such that the ridges of honeycomb cells are aligned to create a plurality of voids enclosed by the ultrathin ALD film on all sides. In this manner, a composite structure having insulating properties can be achieved. Additionally or alternatively, two or more structures can be stacked with an adhesive to form a bulk material. For example, an organic polymer layer can be disposed between two ALD structures to increase the strength and toughness of the resulting composite material. The polymer layer can have a thickness approximately equal to that of the ALD structures. Additionally and/or alternatively, graphene flakes can also be used to adhere the ALD structures. Additionally and/or alternatively, the natural adhesion/stiction that occurs between two contacting ALD layers, for example due to van der Waals forces and/or surface tension-induced forces, can be used to adhere the ALD structures.

Exemplary plate structures are disclosed in U.S. patent application Ser. No. 15/456,718 which is hereby incorporated by reference in its entirety.

In some embodiments, additionally or alternatively, the plate structure can be coupled to detachable tubing. The plate structure can include one or more restraints, anchors, straps, buckles, elastic bands, tape, or any other suitable securing features, including geometric modifications aimed at securing the tubing to the plate. For example and not a limitation, the plate structure coupled to the detachable tubing can be inserted into an eye.

In certain embodiments, the plate structure and a tubing can be implanted separately. For example, at least one tubing can be implanted into an eye to divert a portion of intraocular fluid to an external reservoir created by the plate structure without direct contact with the plate structure. In non-limiting embodiments, the plate structure can be implanted into an eye without tubing. For example, the implanted plate structure can extend into the anterior chamber. The plate structure can be placed beneath the conjunctiva of the eye partially creating an external reservoir beneath the conjunctiva or permitting aqueous humor drainage directly beneath the conjunctiva or Tenon's capsule. The plate structure can be inserted into the anterior chamber of the eye to create a drainage pathway from the anterior chamber to the external reservoir beneath the conjunctiva of the eye or to create a drainage pathway from the anterior chamber to the space directly beneath the conjunctiva or Tenon's capsule. In some embodiments, the plate structure can have a tapered proximal end leading into the anterior chamber of the eye.

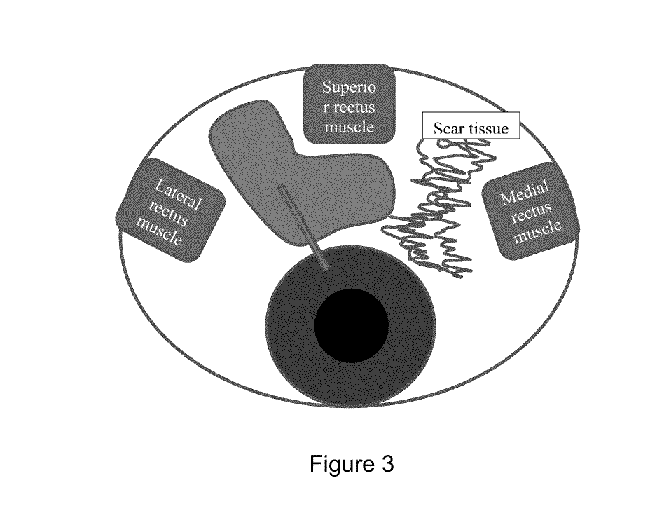

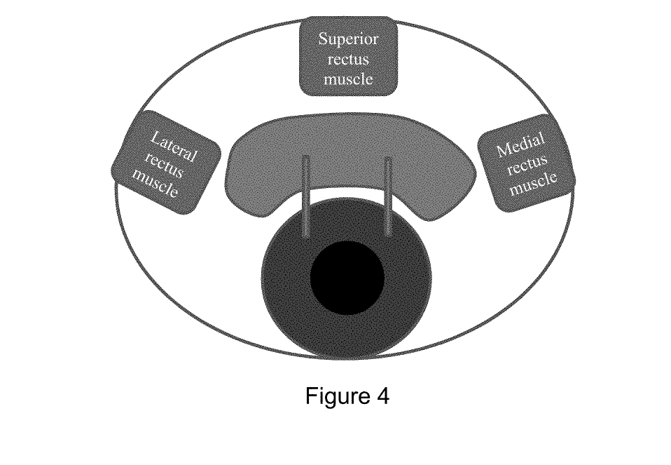

In accordance with the principles of the present invention, the plate structure can have a variety of geometry adapted based on an eye condition of a subject. For example, the geometry can include a mushroom shape, circular shape, elliptic shape, oblong shape, oval shape, or butterfly shape with lateral wings. In some embodiments, the plate structure can have a tapered proximal end leading into the anterior chamber of the eye. The overall size can be modified. Shape and size variations can be prefabricated in the manufacturing process or alternatively applied at the time of surgery but the surgeon or an assistant. In some embodiment, the plate structure can be customized to anatomy and intraocular pressure goals of an individual eye through shape variations. For example, as shown in FIG. 3, the plate structure can be tailored to fit in subconjunctival space away from a scar tissue. The tubing can be inserted to allow egress of aqueous under or above the plate structure. As shown in FIG. 4, plate structure with increased surface area can be used for broad filtration. In some embodiments, the plate structure can be perforated and permeable to oxygen.

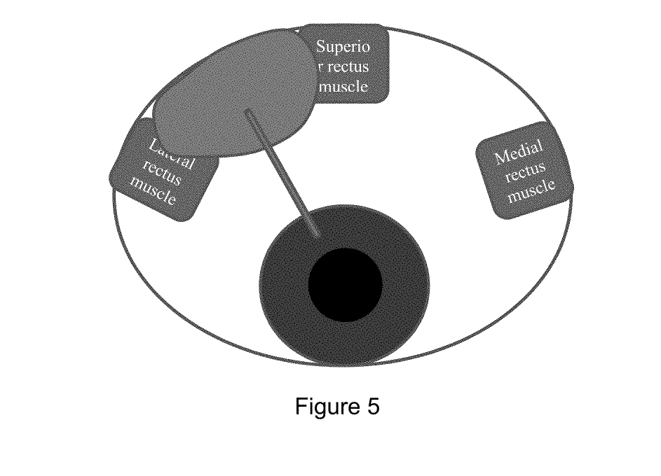

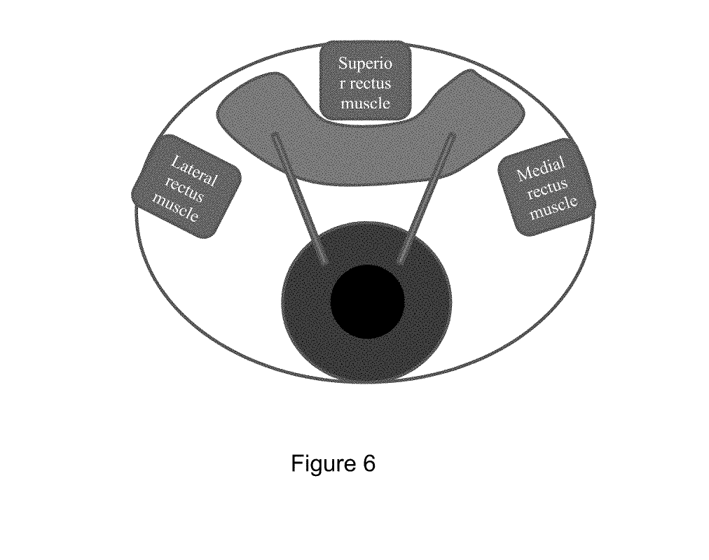

In certain embodiments, the tubing can be modified depending on an eye condition of a subject. For example, as shown in FIGS. 5 and 6, the shape, length, and diameter of the tubing can be adjusted to obtain desired liquid penetration and filtration. In some embodiments, more than one tubing can be inserted to allow increased aqueous filtration and penetration as shown in FIG. 6.

In non-limiting embodiments, the disclosed subject matter provides methods for lowering intraocular pressure using the drainage device. The method can include securing the plate structure to an eye of the subject and inserting the tubing into an anterior chamber of the eye to divert a portion of fluid from inside of the eye to an external reservoir created by the plate structure. For example, a conjunctival incision can be created to allow adequate exposure for insertion of the plate structure. The plate structure can be placed between a sclera and a conjunctiva of the eye. The plate structure can be anchored between superior rectus muscle and lateral rectus muscles with the anterior edge posterior to the limbus. The tubing can be inserted into an anterior chamber of the eye through a limbal region, a corneo-limbal region, or an anterior scleral region of the eye. In certain embodiments, more than one drainage device can be implanted into one or multiple quadrants of the eye.

In certain embodiments, a surgical insertion tool can be used to implant a drainage device into an eye for delivering the drainage device to the precise location. The surgical insertion tool can include specialized forceps, injection devices and device insertion glides. The insertion tools can lead to optimization of placement position, reduced surgical time, reduced number and size of incisions, reduced risk of infection, reduced likelihood of error and increased likelihood of surgical success.

In non-limiting embodiments, the methods can include treating any types of ophthalmic symptoms related to intraocular pressure. Non-limiting examples of ophthalmic symptoms include primary open-angle glaucoma, normal-tension glaucoma, ocular hypertension, primary angle closure glaucoma, congenital and juvenile glaucoma, and secondary glaucoma, including exfoliative, uveitic, neovascularity, traumatic, pigmentary and other secondary glaucoma. In particular, the ophthalmic symptoms include glaucoma and all subtypes of glaucoma.

The following Examples are offered to more fully illustrate the disclosure, but are not to be construed as limiting the scope thereof.

### Example: Single Dose Ocular Tolerability Study in New Zealand White Rabbits

This Example illustrates the use of the plat structure to lower intraocular pressure and the tolerability of the plate structure when implanted beneath the conjunctiva in New Zealand White Rabbits.

Materials and Methods

Fabrication of the Device:

Honeycomb structures were designed and fabricated out of 53 nm-thick ALD alumina (Al₂O₃) and silica (SiO₂). The honeycomb structures were fabricated in different geometries with lateral dimensions varying between 0.5 and 15 millimeters. Three clamping configurations have been used: cantilevers, doubly clamped beams, and rectangular plates clamped on all four sides.

The fabrication started with a double side polished Si wafer. SiN films with a thickness of 180 nm were deposited on both sides using PECVD. Honeycomb structures with a height of 10 μm were patterned in silicon using photolithography and reactive ion etching (RIE). The back side was patterned via photolithography and the openings were obtained by RIE etching of SiN. The SiN mask was removed from the front side and the ALD layer was then deposited. For alumina deposition, trimethylaluminum (TMA) and water were used as precursors and two different temperatures, 150° C. and 250° C., were used. The deposition rates were measured using an ellipsometer to be 0.91 Å/cycle at 150° C. and 1.18 Å/cycle at 250° C.

In order to pattern the ALD layer, a thick layer of SPR 220 resist was spin-coated on the structure. The thickness of the resist was measured to be 14 μm. After the spin coating and soft baking at 105° C. the wafer was cooled down slowly to make sure the photoresist did not crack. After photolithography, inductively coupled plasma etching (ICP) with a BCl3-based chemistry process was used to pattern the alumina ALD layer. In contrast, RIE was used to pattern the silica ALD layer. Anisotropic KOH etching was next. Before placing the wafer in KOH, the top surface was covered with ProTek to prevent the ALD layer from being etched in the KOH solution. A silicon etching rate of 75 μm/hour was measured at 80° C. in the 30% KOH solution. By accurately timing the KOH etching process, it was possible to stop the process ˜20 μm from the top surface. The exact depth was measured using a Zygo profilometer. After that, the ProTek layer was removed and oxygen plasma was performed to make sure the surface of the wafer was completely clean and without any polymer residue. In some embodiments, as an alternative to KOH etching, the silicon substrate can be partially removed using a laser micromachining system such as IPG Photonics IX-280-DSF. XeF₂ etching was used for the final release of the structure. Approximately 100 cycles (30 sec each) of XeF₂ etching with a ratio of 3.2:2 (XeF₂:N₂) was required for the complete release of the structures. Prior to the surgery, a geometry of the plate structure was modified and the tubing was primed with balanced salt solution to confirm patency of the tubing.

Surgical Methods:

Three experimentally naive New Zealand White rabbits (1 male and 2 females), approximately 5 months old and weighing 2.8 to 3.3 kilograms for males and females at the outset of the study were assigned to treatment groups as shown in the table below.

TABLE I
Experimental group

| | | | Number | |
|---|---|---|---|---|
| | | | of Animals | |
| GROUP | Left Eye | Right Eye | Male | Female |
| 1. Test Article | Insertion between the | Sham incision | 1 | 2 |
| | sclera and the | in the | | |
| | conjunctiva via a | conjunctiva | | |
| | conjunctival incision | | | |

To implant the nanomaterial, each rabbit was anesthetized with a combination of Ketamine (40 mg/kg) and Xylazine (4 mg/kg) subcutaneously. Anesthetics were supplemented as needed. All drug usage was documented in the raw data. A few drops of 1% proparacaine (topical anesthesia drops) were placed in each eye at this time as well. Once anesthetized, the rabbit was placed in lateral recumbency and the area surrounding the eye prepped with a Swapstick containing 10% Providone-iodine. The eye was then rinsed with 0.9% sterile saline and another few drops of proparacaine given. A sterile drape was placed over the rabbit allowing exposure of the eye. Sterile instruments (steam autoclaved prior to first procedure and then chemically sterilized in chlorhexidine solution and rinsed with sterile water/saline between animals). Sterile gloves were worn.

The eyelid was kept open manually or with an eyelid speculum for the procedure. The eye was rotated medially using Colibri forceps and a small incision made in the conjunctiva lateral to the iris. A subconjunctival pocket was created ventrally and the nanomaterial placed within. Upon placement, the eye was allowed to rotate back to normal position and placement of the nanomaterial was observed to assure it is lying well within the subconjunctival pocket. The rabbit was then rotated to the other side and a sham procedure performed similarly on the contralateral eye, with no nanomaterial or other material implanted. Sterile ophthalmic ointment was placed on both eyes during the recovery period.

Observations and Measurements:

The nanomaterial was implanted into the eye of the subjects on Day 1 via a conjunctival incision between the sclera and the conjunctiva. Mortality and clinical observations were evaluated daily. Ocular irritation scores were recorded prior to dose on Day 1, once daily on Days 2-5, Day 12, and Day 19. Body weights were recorded weekly. Food consumption was recorded daily. All animals were sacrificed on Day 21. The eyes with optic nerve for all animals were harvested at necropsy and evaluated microscopically.

Histological analysis: The animals were sacrificed with an overdose of an intravenous barbiturate on Day 21. All animals were necropsied. The eyes with optic nerve were collected and immediately fixed in Davidson's fixative for 24-48 hours. After the nerve specimens were dehydrated with increasing concentrations of ethanol (30-100%), the nerves were sectioned with a sharp razor blade. The sections were then embedded in paraffin in descending order and sectioned at 3 um in thickness. The sections were stained with hematoxylin and eosin. Two sections (halves of the globe) with pupillary-optic disc orientation were trimmed from each eye, and two levels were microtomed per paraffin block, resulting in four slides per eye available for microphonic examination,

Results and Discussion

One male and two females New Zealand White rabbits were dosed once on Day 1 with the nano material via a conjunctival incision between the sclera and the conjunctiva.

Mortality/Morbidity:

There were no early deaths during the study. All animals survived until their scheduled sacrifice on Day 21.

Clinical Observations:

On Day 1, mild to moderately decreased behavioral activity was noted post-surgery, and all animals had closed or partially closed eyes at 2-4 hours post-dose. These findings were considered test article unrelated and were secondary to the anesthesia and surgical procedures. All animals appeared normal on study days 2-21, and no clinical signs were noted.

Ocular Observations:

The eyes of all animals (left and right) had ocular Draize scores of 0 prior to dosing on Day 1. Minimal overall Draize scores were recorded on study Days 2 and 3. Scores were noted in both, the left and right eye (plate structure implant and sham procedure, respectively). By Day 4, no ocular scores were noted. Table II below, summarizes the overall ocular Draize scores recorded during the study.

TABLE II
Draize Scores recorded during the study

| | Overall Ocular Draize Score | | | | | |
|---|---|---|---|---|---|---|
| | Left Eye | | | Right Eye | | |
| | Animal #s | | | Animal #s | | |
| | 1414 | 1413 | 1416 | 1414 | 1413 | 1416 |
| Study Day 1 | 0 | 0 | 0 | 0 | 0 | 0 |
| Study Day 2 | 0 | 2 | 2 | 0 | 2 | 0 |
| Study Day 3 | 0 | 2 | 2 | 0 | 0 | 2 |
| Study Day 4 | 0 | 0 | 0 | 0 | 0 | 0 |
| Study Day 5 | 0 | 0 | 0 | 0 | 0 | 0 |
| Study Day 12 | 0 | 0 | 0 | 0 | 0 | 0 |
| Study Day 19 | 0 | 0 | 0 | 0 | 0 | 0 |

Body Weight:

No apparent test article-related effects on body weight or body weight gains were noted.

Food Consumption:

There were no test article-related effects on food consumption. The animals ate all their food on essentially all days.

Postmortem Observations

Gross Necropsy Findings:

No gross necropsy findings were noted at scheduled sacrifice on Day 21.

Histopathology:

The nanomaterial was not visible microscopically in any animal. A focal scleral alteration was noted near the limbus in several eyes, consisting of an elevation and separation of conjunctiva and superficial collagen fibers from the deeper collagen fibers of the sclera, creating an empty space. Aside from fragmentation of collagen, no notable tissue reaction was evident. While alterations of minimal (Grade 1) severity was noted in two control (right) eyes, alterations of mild (Grade 2) to moderate (Grade 3) severity were evident in two out of three treated (left) eyes, rising suspicion that the tissue alterations in the eyes receiving the nanomaterial could at least in part represent implant sites in which the implant fragmented or washed out during processing. Conjunctival hyperplasia, lymphoplasmacytic infiltrate, and/or fibrosis of minimal severity were noted near the limbus in right and left eyes of all three animals. These lesions can be explained as spontaneous background findings and/or associated with surgical manipulation.

In summary, no test related clinical observations, effects on body weight or body weight gains, or effects on food consumption were noted. Post-surgery, overall Draize scores were minimal and all eyes appeared normal by Day 4. No gross necropsy findings were noted at scheduled sacrifice on Day 21. The nanomaterial was not visible after tissue processing, and no tissue reaction was noted at the implant site. In conclusion, the nanomaterial when implanted beneath the conjunctiva in New Zealand White rabbits was well tolerated.

It will be understood that the foregoing only demonstrates the tolerability of a nanomaterial when implanted into the eye and is only illustrative of the principles of the present disclosure, and that various modifications can be made by those skilled in the art without departing from the scope and spirit of the present disclosure.

What is claimed:

1. A method for lowering intraocular pressure of a subject using a drainage device including a plate structure having a geometry including a thickness between about 1 nm and about 1000 nm comprising:
modifying the geometry of the plate structure based on an eye condition of the subject; and
securing the plate structure to an eye of the subject.

2. The method of claim 1, wherein the securing further comprises placing the plate structure between a sclera and a conjunctiva of the eye.

3. The method of claim 1, further comprising inserting a tubing into an anterior chamber of the eye to divert a portion of fluid from inside the eye to the plate structure, whereby the plate structure forms an external reservoir.

4. The method of claim 3, wherein the inserting comprises inserting the tubing into an anterior chamber of the eye through a limbal region, a corneo-limbal region, or an anterior scleral region of the eye.

5. The method of claim 3, wherein the inserting comprises modifying a geometry of the tubing based on the eye condition of the subject.

6. The method of claim 5, wherein the geometry of the tubing is configured to adjust fluid penetration and/or filtration.

7. The method of claim 1, wherein the plate structure comprises a biocompatible material selected from the group consisting of aluminum oxide, hafnium oxide, silica, titanium nitride, titanium carbide, a derivative thereof, and a combination thereof.

8. The method of claim 3, wherein the tubing comprises a biocompatible polymer.

9. The method of claim 1, wherein the lowering intraocular pressure is configured to treat glaucoma induced visual deterioration.

10. The method of claim 1, further comprising implanting the drainage device into the eye using an insertion device.

11. The method of claim 1, wherein the plate structure is configured to extend into an anterior chamber of the eye.

12. The method of claim 11, wherein the plate structure is configured to create a drainage pathway from the anterior chamber to an external reservoir formed by the plate structure or to create the drainage pathway from the anterior chamber to a space beneath a conjunctiva or a Tenon's capsule.

13. A system for lowering intraocular pressure of a subject comprising:
a plate structure for creating an external reservoir on a surface of an eye, the plate structure having a thickness between about 1 nm and about 1000 nm,
wherein the plate structure comprises a plate structure having a geometry adapted based on an eye condition of the subject.

14. The system of claim 13, wherein the plate structure comprises:
at least one base plate in a first plane; and
a plurality of out-of-plane rib plates forming at least one strengthening rib,
wherein the at least one strengthening rib is formed such that there is no straight line path extending through the height or width of the plate structure that does not intersect the at least one base plate and the at least one strengthening rib.

15. The system of claim 13, wherein the plate structure comprises a biocompatible material selected from the group consisting of aluminum oxide, hafnium oxide, silica, titanium nitride, titanium carbide, a derivative thereof, and a combination thereof.

16. The system of claim 13, further comprising a tubing, adapted for diverting a portion of intraocular fluid to the external reservoir.

17. The system of claim 16, wherein the tubing comprises a biocompatible polymer.

18. The system of claim 13, further comprising an insertion device adapted for implanting the plate structure into the eye.

19. The system of claim 13, wherein the plate structure is configured to extend into an anterior chamber of the eye.

20. The system of claim 19, wherein the plate structure is configured to create a drainage pathway from the anterior chamber to an external reservoir formed by the plate structure or to create the drainage pathway from the anterior chamber to a space beneath a conjunctiva or a Tenon's capsule.
